(19) United States
(12) Patent Application Publication
(10) Pub. No.: US 2018/0221687 A1
(43) Pub. Date: Aug. 9, 2018

(54) HAIR GROWTH STIMULATION

(71) Applicant: KONINKLIJKE PHILIPS N.V., EINDHOVEN (NL)

(72) Inventors: KIRAN KUMAR THUMMA, EINDHOVEN (NL); YANNYK PARULIAN JULIAN BOURQUIN, EINDHOVEN (NL)

(21) Appl. No.: 15/747,922

(22) PCT Filed: Jul. 28, 2016
(86) PCT No.: PCT/EP2016/068074

§ 371 Date: Jan. 26, 2018

(30) Foreign Application Priority Data
Jul. 29, 2015 (EP) 15178818.9

(51) Int. Cl. A61N 7/00

(52) U.S. Cl. CPC A61N 7/00; A61N 2007/0034

(57) ABSTRACT

Hair growth stimulating device to obtain a non-chemical hair growth stimulation comprising an ultrasound transducer (UST), and a driver (DE) for the ultrasound transducer, wherein the driver is arranged for having the ultrasound transducer (UST) generate an ultrasound signal having a frequency not exceeding 100 kHz, preferably 20 kHz, at a power not exceeding 500 mW/cm², preferably 250 mW/cm². The frequency may be subject to a frequency sweep not exceeding 20%, preferably 10%. Pulses having a pulse width between 10 ms and 500 ms may be used.

### FIELD OF THE INVENTION

The invention relates to hair growth stimulation.

### BACKGROUND OF THE INVENTION

Hairs at different body locations have specific length. The length of the hairs is mainly define by two factors which are the duration of the anagen phase (growing phase of the hair) and the growth rate. Most of the products in the market are topical based or light based solutions for stimulating hair growth. There are many patents relating to hair growth and hardly any on making the existing hairs grow faster.

US2011230793 discloses a method for the treatment of alopecia and in particular the problem of male-pattern baldness. By application of controlled ultrasound exposures to specific regions in the skin tissue, a physiological response will develop which causes cells in the hair follicle to increase in number, and/or new follicles to form or multiply in response to controlled damage to existing cells, leading to new hair follicles and/or increased shaft thickness in existing hair follicles resulting in more robust hair growth. Preferably, the applicator utilizes a transducer capable of delivering high intensity ultrasound pulses of varying pulse lengths ranging from microseconds to milliseconds, with acoustic intensities at the focus ranging from a few thousand to tens of thousands of W/cm², and with acoustic frequencies ranging from a few MHz to approximately 20 MHz.

DE102008015486 discloses a hair device for stimulating hair growth that uses brush elements fed with ultrasound (i.e. beyond 20 kHz) to transfer vibrations onto the skin of a user's head. The scalp is stimulated so that blood circulation is enhanced and the penetration of active ingredients is promoted through the epidermis to the hair roots.

US 2011/0269693 discloses ultrasound delivery apparatus comprising flexible arrays of transducers and to methods and topical compositions for the treatment of skin, in particular for the treatment of cosmetic skin conditions and to improve the appearance of sun damaged and/or aged skin. The composition may comprise one or more anti-glycation agent, one or more antioxidant, a dermatologically acceptable excipient and optionally one or more substance capable of inducing expression of a molecular chaperone.

### SUMMARY OF THE INVENTION

It is, inter alia, an object of the invention to provide an improved hair growth stimulation without using chemicals such as the active ingredients and/or topical compositions known from some prior art documents. The invention is defined by the independent claims. Advantageous embodiments are defined in the dependent claims.

One aspect of the invention provides a hair growth stimulating device comprising an ultrasound transducer, and a driver for the ultrasound transducer, wherein the driver is arranged for having the ultrasound transducer generate an ultrasound signal having a frequency not exceeding 100 kHz, preferably 20 kHz, at a power not exceeding 500 mW/cm², preferably 250 mW/cm². The frequency may be subject to a frequency sweep not exceeding 20%, preferably 10%. Pulses having a pulse width between 10 ms and 500 ms may be used.

These and other aspects of the invention will be apparent from and elucidated with reference to the embodiments described hereinafter.

### BRIEF DESCRIPTION OF THE DRAWINGS

FIG. 1 shows an embodiment of a hair growth stimulating device in accordance with the present invention.

### DESCRIPTION OF EMBODIMENTS

Embodiments of the invention provide an ultrasonic treatment of hair whereby a larger area of skin/hair can be treated in order to make the hairs grow faster, whereby the total treatment time can be decreased. Faster hair growth by means of ultrasound using the parameters described within this invention is done not by heating the hair follicles but by non-thermal mechanisms which are the induced chemical/biological reactions by application of ultrasound. This removes the heating effects on the skin surface and also on the hair follicles.

Experiments have been done on ex-vivo skin with hair follicles. After the ultrasound treatment, the hairs are carefully isolated and cultured to see the growth of the hairs by taking the pictures every day. After 5 days of culturing the treated hairs, the hairs remain healthy. Moreover, the treated hairs grow 20%-30% faster than the untreated hairs. Statistical analysis shows significant difference between control and treated hairs. The ultrasound stimulates the faster hair growth by a direct stimulation of cells resulting in an increase of proliferation.

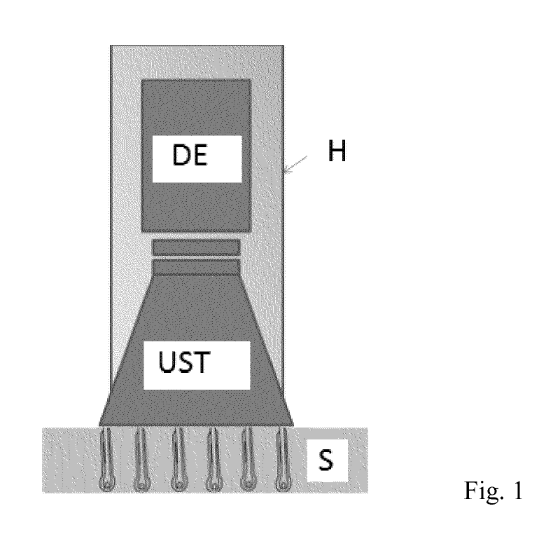

FIG. 1 shows an embodiment of a hair growth stimulating device in accordance with the present invention. A housing H comprises driver electronics DE and an ultrasound transducer UST, which are used on skin S having a plurality of hair follicles.

The device can be a single transducer which can be applied on the skin or in another embodiment, it can be an array of transducers. The array—which can treat a larger area—is made possible because of the limited heating of the skin using the parameters in accordance with the present invention.

Many settings have been tried in the lab on ex-vivo skin with grey hairs and black hairs. The desired faster hair growth can be achieved with the parameters given in the following table:

| | |
|---|---|
| Frequency | 20 kHz |
| Sweep bandwidth | 2 kHz |
| Sweep time | 1 s |
| Power | 100 mW/cm²-500 mW/cm² |
| Treatment time | 10 s-1 minute |

Using these parameters, we have measured the temperature increase at a few millimeters below the skin where the hair follicles are present. The temperature increase was between 3-5° C. for a treatment time of 1 minute. Such limited temperature increase will not result in discomfort for the user of the device. Additionally, this has been confirmed from simulations where we simulated the experimental setup with a transducer and the skin and calculated the temperature rise. The simulations showed an increase in temperature of 3° C. for a treatment time of 1 minute.

Further experiments were carried out at lower power levels of 500 mW/cm² and 250 mW/cm² using the otherwise the same parameters as described in table 2. The hair growth is faster at 250 mW/cm² than at 500 mW/cm². Above 1 W/cm² at a treatment time of above 10 s, no significant faster hair growth is measured.

The invention thus provides an ultrasonic hair stimulation device with specific advantages of large treatment area on different skin and hair types with favorable power levels and short treatment times. In an embodiment, specific frequencies (20-22 kHz with sweep) and power (between 100-500 mW/cm2) and a treatment time of 1 min are used. A pulse width of 10 ms or higher is used. Advantageously, no treatment medium is used.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. The mere fact that certain measures are recited in mutually different dependent claims does not indicate that a combination of these measures cannot be used to advantage.

1. (canceled)

2. (canceled)

3. (canceled)

4. (canceled)

5. (canceled)

6. Hair growth stimulating method to obtain a non-chemical hair growth stimulation, the hair growth stimulating method comprising:
generating an ultrasound signal having a frequency not exceeding 100 kHz at a power density not exceeding 500 mW/cm², and
applying the ultrasound signal to hair follicles.

7. Hair growth stimulating method as claimed in claim 6, wherein the frequency is between 20 and 30 kHz, and preferably between 20 and 25 kHz, more preferably 20 kHz.

8. Hair growth stimulating method as claimed in claim 6, wherein the frequency is subject to a frequency sweep not exceeding 20%, preferably 10%.

9. Hair growth stimulating method as claimed in claim 6, wherein pulses having a pulse width between 10 ms and 500 ms are used.

10. Hair growth stimulating method as claimed in claim 6, wherein the power density is between 100-500 mW/cm², and preferably 250 mW/cm².

11. Hair growth stimulating method as claimed in claim 6, wherein ultrasound is applied during a period between 0.5 and 2 minutes, and preferably during about 1 minute.

12. Hair growth stimulating device to be used for carrying out the method as claimed in claim 6 to obtain a non-chemical hair growth stimulation, the hair growth stimulating device comprising:
an ultrasound transducer; and
a driver for the ultrasound transducer, wherein the driver is arranged for having the ultrasound transducer generate an ultrasound signal having a frequency not exceeding 100 kHz at a power density not exceeding 500 mW/cm².

13. Hair growth stimulating device as claimed in claim 12, wherein the frequency is between 20 and 30 kHz, and preferably between 20 and 25 kHz, more preferably 20 kHz.

14. Hair growth stimulating device as claimed in claim 12, wherein the frequency is subject to a frequency sweep not exceeding 20%, preferably 10%.

15. Hair growth stimulating device as claimed in claim 12, wherein pulses having a pulse width between 10 ms and 500 ms are used.

16. Hair growth stimulating device as claimed in claim 12, wherein the power density is between 100-500 mW/cm², and preferably 250 mW/cm².

17. Hair growth stimulating device as claimed in claim 12, wherein ultrasound is applied during a period between 0.5 and 2 minutes, and preferably during about 1 minute.
